(19) United States
(12) Patent Application Publication
(10) Pub. No.: US 2012/0269780 A1
(43) Pub. Date: Oct. 25, 2012

(54) Method For Preparing an Acellular Organic Tissue of Human or Animal Origin For Revitalization

(75) Inventor: Maurizio Marzaro, Treviso (IT)

(73) Assignee: TELEA BIOTECH S.R.L., Sandrigo (IT)

(21) Appl. No.: 13/504,183

(22) PCT Filed: Oct. 29, 2010
(86) PCT No.: PCT/IB2010/002767

§ 371 Date: Jul. 3, 2012

(30) Foreign Application Priority Data
Oct. 29, 2009 (IT) VI2009A000263

(51) Int. Cl. A61K 35/12

(52) U.S. Cl. 424/93.7; 424/520

(57) ABSTRACT

The invention concerns a method for the preparation of an acellular organic tissue for revitalization, in particular for the introduction of living cells, comprising a stage in which the acellular organic tissue (2; 12) is provided with a plurality of holes (4; 14) made through its surface (8; 18) and extending towards the inside of the tissue (2; 12). The holes intersect partially, thus forming holes (4; 14) partially communicating with each other. The invention also concerns the acellular organic tissue obtained with this method.

### TECHNICAL BACKGROUND OF THE INVENTION

The invention concerns a method for the preparation of an acellular organic tissue for revitalization, and in particular for the introduction of living cells, comprising a stage in which the surface of an acellular organic tissue is provided with a plurality of holes extending towards the inside of the tissue. The invention also concerns an acellular organic tissue provided with a plurality of holes extending from the outer surface of the tissue towards the inside of the tissue, wherein said tissue can be obtained using the method that is the subject of the invention.

### STATE OF THE ART

As is known in the medical sector, and more specifically in the field of surgery, it is increasingly important to be able to use tissues that can be implanted in living beings in order to meet the increasing need to replace parts of organs or whole organs.

The production of biological substitutes that are prepared in laboratories and then implanted in the host, be it an animal or human being, belongs to the medical technique and is known as tissue engineering.

According to a known technique, the preparation of tissues to be transplanted is carried out in the laboratory by inserting cells into a matrix consisting of an inorganic support generally called “scaffold”.

The “scaffold”, inserted in order to replace a defect in the organ to be treated, favours the three-dimensional organization of the cells until the tissue is completely formed.

Obviously, said scaffold must undergo a degradation process until it disappears completely and is replaced by the regenerated tissue, this process being favoured by the cells grafted in said scaffold.

The transplants obtained with this system can be obtained with both artificial scaffolds and natural scaffolds (from a “donor”) that can derive from humans and animals, for example the esophageal wall.

In order to be able to use a scaffold deriving from a donor and then transplant it in a human host, it is necessary to previously treat the tissue in such a way as to completely eliminate all the cells present between the connective tissue fibres and successively reintroduce human cells belonging to the host, so as to avoid rejection reactions.

The techniques for making a scaffold, that is, an acellular matrix starting from tissues taken from a donor, are known and therefore will not be exhaustively described herein, as they substantially consist in the immersion of the tissue to be treated in liquids containing enzymatic substances capable of digesting and destroying the living cells contained in the tissue, while at the same time maintaining the connective tissue fibres intact.

Once an acellular tissue, ready to receive the cells taken from the host, has been obtained, said tissue or scaffold is placed in a so-called “Petri capsule”, a small container used in every biology lab, on the bottom of which the tissue to be revitalized is placed, or in an analogous device.

Revitalization takes place through the introduction of the host's stem cells grown in a culture medium that allows cell nutrition, life, multiplication and diffusion.

Substantially, the stem cells, which at the beginning rest on the upper surface of the tissue, move through the natural interstices of the latter, interstices that were previously occupied by the donor's cells.

After a given lapse of time, with controlled temperatures and in the presence of nutritional substances contained in the culture medium, the living cells are repositioned in the interstices of the tissue which becomes ready to be transplanted in the host's organ.

It can be noticed that generally the cells used for revitalizing the scaffold are stem cells that will differentiate, or have already differentiated, and will assume the specific function of the organ in which the revitalized tissue will be grafted.

The success or failure of the transplant of the tissue treated as described depends on the capillary diffusion of the cells inside the tissue. If said diffusion takes place with difficulty, or superficially and not in depth, the grafted tissue cannot be revitalized and tends to necrotize, which means that the transplant will fail.

The above clearly shows that an essential and important condition to be met, which practically cannot be renounced, is the in-depth revitalization of the tissue, in all its parts and above all through its entire depth.

At present, even if the preparation and revitalization treatment takes place for a sufficiently long time, certain results guaranteeing the success of the transplant cannot be obtained.

This is due to the insufficient in-depth penetration of the living cells that must be grafted in the scaffold. Practically, this drawback considerably limits the possibility of preparing tissues suitable for transplants, as rather thick tissues, not being able to be penetrated in depth, are not revitalized completely after the transplant. It is evident, therefore, that the technique described above is suitable only for transplanting tissues with very limited thickness, for example not exceeding approximately 0.1 mm.

In order to overcome this drawback, document WO 2008/146106 A2 filed in the name of the present applicant describes a method according to which the acellular organic tissue is provided with holes in order to favour the diffusion of the cells through all the tissue. U.S. Pat. No. 5,112,354 describes the perforation of a bone part intended to increase the surface exposed to the demineralizing agents and favour the interaction with mesenchymal cells introduced for osteoinduction. According to the mentioned documents, notwithstanding a considerable improvement compared to the traditional techniques, the simple perforation of the tissue with the purpose of preparing it for the introduction of cells still takes a relatively long time to achieve a uniform penetration of the cells, in particular in case of very thick and extended tissues.

### DESCRIPTION OF THE INVENTION

The object of the present invention is to provide an acellular organic tissue ready to be colonized by living cells and capable of overcoming the drawbacks mentioned above.

More specifically, it is the object of the invention to provide an acellular organic tissue such that, when said tissue is revitalized with stem cells, compared to the state of the art, it is easier for the cells to enter and penetrate every single interstice in the mesh formed by the fibres of the connective tissue, so as to substantially reproduce the conditions of the tissue prior to devitalization.

It is also the object of the invention to obtain a further significant and considerable reduction in the treatment time necessary for revitalizing the acellular scaffold when the living cells intended to prepare the tissue for the transplant are introduced.

Therefore, it is one object of the invention to develop a method for producing an acellular organic tissue prepared in an improved form for the uniform and quick introduction of revitalizing cells.

The objects of the invention are achieved by the method of the type described at the beginning, in which the holes, extending from the outer surface of the tissue towards the inside of the same, intersect at least partially, thus forming holes that are at least partially in communication with each other and producing a tissue provided with a plurality of holes extending from the surface of the tissue towards its inside, so that said holes are at least partially in communication with each other. In other words, the tissue that can be obtained with the method of the invention is provided with a plurality of holes, and a single hole communicates along its extension with one or more of the other holes, thus forming a reticular structure. The mesh of communicating holes considerably increases the speed with which they can be filled with regenerating cells. Furthermore, the reticular structure favours the uniform diffusion of the cells. In the simplest case, said communicating holes are substantially straight, being made with needles through the outer surface of said tissue, so that the holes intersect.

Said holes are made in such a way that their depth extends at least partially into the thickness of the tissue, but preferably into almost the full thickness of the tissue or, in particular cases, into the full thickness of the tissue. A thin non perforated layer is preferably left on the side opposite the perforated surface in order to prevent the introduced cells from flowing out.

The holes can be made through the thickness of the tissue to be treated with various devices and methods, provided that the execution of said holes does not cause any deterioration or alteration (tears, necrosis, decrease or increase in thickness, alteration of the liquid content, coagulation) of the connective tissue around the hole and in any case the scaffold in general.

According to a particularly advantageous embodiment of the invention, the tissue is also provided with reservoir cavities communicating with at least part of said holes. The reservoir cavities can be considered as recesses, that is, as deposits for reserves of cells to be introduced in the tissue, comparable to deposits of reserve cells in the body (for example, deposits for epithelial cells of the intestine). If the cells to be introduced in the tissue are no more simply applied to the surface of the tissue, but also introduced in the reservoir cavities made in the tissue that communicate with at least part of the holes provided, it is possible to further improve the uniformity of distribution of the cells and to further accelerate the penetration of the cells throughout the tissue.

The creation of reservoir cavities communicating with at least part of said holes increases, for the cells applied to the tissue (and introduced in the reservoir cavities), the number of access points to the mesh of holes.

Advantageously, the reservoir cavities are on the surface of the tissue in order to make it easier to fill them with cells, that is, to ensure easy access to them. The diameter of the holes produced, in any case, must be sufficient to allow the cells to easily get into said holes and revitalize the surrounding tissue.

Regarding the size of the cells, their diameter should be at least 50 μm. The selection of the diameter must also be made according to the need to maintain the structural integrity of the tissue, at the same time ensuring quick penetration and complete filling of the holes.

The reservoir cavities can be prepared before or after making the holes. The channels are preferably applied after the creation of the reservoir cavities, so as to ensure the stability of the tissue.

The reservoir cavities are substantially cylindrical in shape, with diameter up to approximately 1 mm. Reservoir cavities with diameters from 700 μm to 1 mm are particularly suitable for this purpose. The substantially cylindrical reservoir cavities can be easily created as holes made with a corresponding needle having a suitable diameter. Obviously, the diameter of the reservoir cavities must not be such as to weaken the structure of the tissue. The diameter of the reservoir cavities should be larger than the diameter of the channels communicating with them, so that the reservoir cavities can serve as cell reserves and that the channels are not transformed in further reservoir cavities that weaken the structure of the tissue itself. The depth of the reservoir cavities depends on the characteristics of the tissue and can be easily optimized according to the needs by a person skilled in the art.

The same applies to the distance between channels and reservoir cavities. Even distances of approximately 200 μm between the single reservoir cavities can be envisaged, as well as longer or shorter distances. The ideal distances must be selected according to the needs in terms of penetration speed and stability of the tissue.

In another preferred embodiment of the invention the holes have diameters different from one another. The same applies to the reservoir cavities, which can even have different diameters inside the tissue. The choice of the distribution of the diameters must be determined by a compromise between the increase in penetration speed, which certainly increases as the diameter increases, and the need to avoid weakening the structure of the tissue too much through excessive perforation. The possibility to vary the diameters of the holes inside the tissue offers higher flexibility in the adjustment of the balance between accelerated penetration and stability of the tissue.

In order to efficiently avoid necrosis problems during the formation of the holes and/or reservoir cavities, said plurality of holes and/or reservoir cavities is preferably made by means of one or more metal needles connected to an electric power source, causing on the tip of each needle the passage of an electric current whose intensity and wave shape are such as to supply a quantity of energy sufficient to open the bonds that join the molecules of the organic tissue in proximity to the tip of said needle, each hole being such that the tip of said needle enters the space left free by the opening of said molecular bonds and makes said hole. It should be understood that the holes and/or the reservoir cavities can be made also in another manner, for example mechanically.

The best results in terms of quality of the holes/reservoir cavities made in the tissue are obtained by applying to the tip of each needle making a hole a high frequency voltage, generally 4 MHz, producing a passage of electric current that is weak but sufficient to open the bonds between the molecules of the connective tissue, so as to make a hole without breaking down the molecules themselves.

The holes and/or reservoir cavities are preferably made using a needle powered with a voltage of preferably 200-230 Volts and the wave frequency of 4 MHz indicated above. Advantageously, the sinusoidal voltage wave applied is a distorted sinusoidal wave, thus with harmonics at least of the first, second and third order.

The current available on the tip of each needle is preferably adjusted so as to be included between 2 and 2.5 mA.

It is logical and evident that making several intersecting holes means creating new paths for grafting cells down to the deep parts of the tissue, which guarantees complete revitalization of the tissue itself.

The preparation of the acellular organic tissue for revitalization may comprise a step for the introduction of various materials, for example washing solutions, nutrition solutions, cells of different types (for example, autologous cells, from a different donor or of a different type, cells to be differentiated or already differentiated) ecc. Advantageously, the introduction of these materials in the holes takes place with the aid of a suction system that comprises a drive mechanism suited to facilitate the entrance of the materials in the holes. The suction system may consist of a peristaltic pump. The tissue prepared for revitalization according to the invention is like a permeable sponge for liquids and along the holes also for cells. In the case where the tissue is provided with through holes and therefore does not have a non-perforated layer on the side opposite the perforated surface, the tissue is arranged on a biodegradable membrane provided with micro pores permeable to liquids but not to cells, thus preventing the cells from flowing out during the suction stage.

Advantageously, the holes are made in the tissue using a needle or an array of needles moved and inclined according to a control system managed by an algorithm that guarantees that the holes are formed so as to intersect with a predetermined density, that is, forming a determined number of points where they cross each other.

The method described above can be used for all types of organic tissues. The acellular tissues provided with communicating holes can be revitalized with all the types of cells suitable for the future use of the tissue.

The tissue of the invention practically poses no limits to the thickness of the tissue to be transplanted, as the presence of communicating holes that can be made through the full thickness of the tissue and over its entire surface guarantees complete revitalization. This is due to the fact that the living cells that will be reintroduced in the acellular scaffold, and preferably in the reservoir cavities, can reach every single part of the tissue.

Also the acellular organic tissue that can be obtained using the method of the invention forms part of the invention. The method provides an acellular organic tissue provided with holes that extend from the surface of the tissue towards its inside, wherein the holes are at least partially communicating with each other. According to the variants of the method described above the tissue may also comprise reservoir cavities. The holes and/or reservoir cavities in the tissue prepared according to the invention have the diameters, depths and distances described above.

Construction variants of the invention are the subject of the dependent claims. Further characteristics and details of the invention will be highlighted in greater detail in the description of preferred embodiments of the invention provided as non-limiting examples.

### BRIEF DESCRIPTION OF THE DRAWINGS

The invention is now illustrated with the aid of the attached drawings, wherein:

FIG. 1 shows a schematic cross-section of the acellular organic tissue prepared for revitalization and provided with communicating holes;

FIG. 2 shows a top view of the acellular organic tissue prepared for revitalization and provided with reservoir cavities and holes;

FIG. 3 shows a cross-section of the tissue of FIG. 2 along line III-III of FIG. 2.

### DESCRIPTION OF THE EXAMPLES OF EMBODIMENT

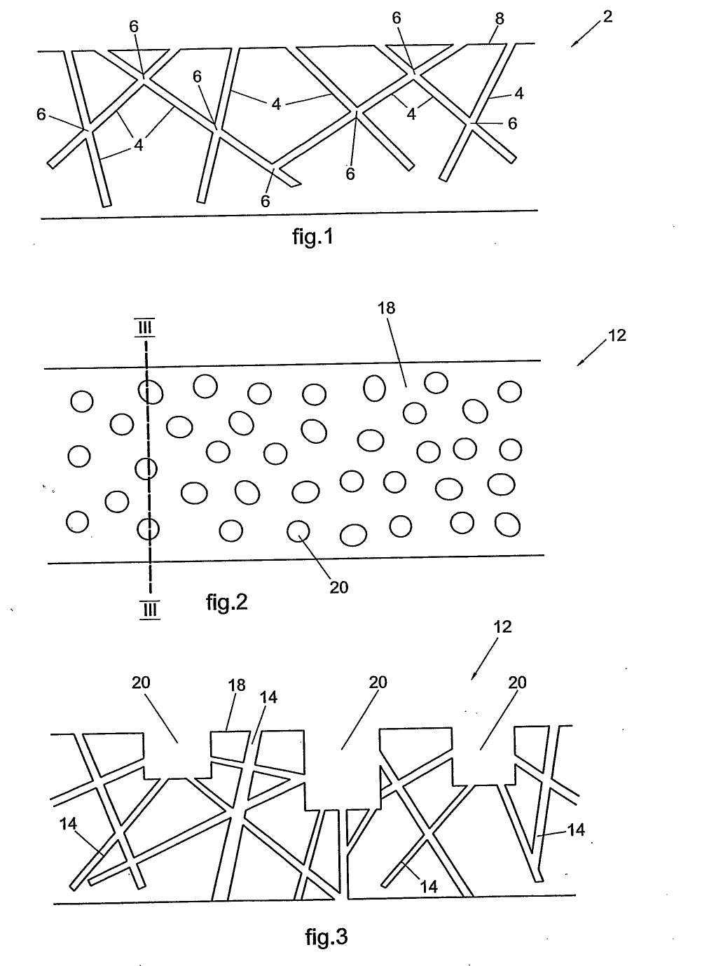

As shown in FIG. 1, an acellular organic tissue indicated as a whole by 2 is provided with a plurality of holes 4 that intersect in the crossing points 6 and are therefore communicating with each other. In the example shown all the holes 4 are inclined with respect to the surface 8 of the tissue 2. However, it is possible to assume that part of the holes is perpendicular to the surface 8 of the tissue 2.

Obviously, the penetration/perforation must be repeated over the entire surface 8 of the scaffold 2 in order to obtain an homogeneous distribution of the holes 4 in the entire thickness and over the entire useful surface of the tissue 2 to be grafted.

FIG. 2 shows a top view of a further embodiment of the invention, in which an acellular organic tissue 12 is provided not only with holes 14 (see FIG. 3, where only some holes are shown for the sake of clarity) but also with reservoir cavities 20. It is possible to observe a plurality of reservoir cavities 20 in the form of large holes with circular cross-section on the surface 18 of the tissue 12.

Finally, FIG. 3 shows in a cross-section along line of FIG. 2 how the holes 14 communicate with each other and with the reservoir cavities 20.

Once the series of holes 4, 14 (and if necessary of reservoir cavities 20) has been made in the acellular tissue 2, 12, as explained above, it is clear that said tissue without cells can be arranged on a Petri capsule or an analogous device, in which the living cells, generally of the stern type, of the future host are now introduced. Said stem cells, properly nourished in a culture medium, can easily and quickly get into all the holes 4, 14 provided, thus guaranteeing a complete and effective revitalization of the whole tissue to be transplanted.

A typical process is structured as follows: an organic tissue 2, 12 without cells and thus previously treated, called scaffold, is preferably laid and spread over a plane surface.

A needle is powered with electric current so as to supply to the molecules of the tissue to be treated with a quantity of energy that is just sufficient to break the bonds between the molecules involved in the passage of current, while the surrounding area is not affected by any tearing or breaking effect, necrosis, decrease or increase in thickness, or alteration of the liquid content, coagulation or other degenerative effect.

Substantially, this opening of the molecular bonds is equivalent to the production of a micro hole 4, 14, that in practice has the same diameter as each needle, considering, in any case, that the minimum diameter of the needle cannot be less than that of the revitalization cells.

The needle is thus introduced slowly enough and so that during its advance its tip finds the hole already prepared by the passage of current and by the consequent breakage of the molecular bonds.

The reservoir cavities 20 can be produced in the same way, by choosing a needle with suitable diameter, being it preferable but not strictly necessary to make the reservoir cavities before the holes.

The formation of a mesh of holes is particularly important and useful as the cells that will be reintroduced can penetrate the tissue in depth and take root in the walls of the hole, multiply and thus revitalize very quickly all the organic tissue.

It can be understood that the method of the invention and the tissue that can be produced therewith achieve all the objects of the invention, as perfect and effective revitalization is obtained, thus eliminating any risk of failure of the transplant that must be carried out. Furthermore, the revitalization process takes place even quicker and with a more uniform cell diffusion than in the known technique.

Upon implementation, further changes or construction variants of the method and tissue of the invention, not described herein, may be carried out. Said changes or construction variants must all be considered protected by the present patent, provided that they fall within the scope of the claims expressed below.

1) Method for preparing an acellular organic tissue of human or animal origin for revitalization, in particular for the introduction of living cells, comprising a step in which said acellular organic tissue is provided with a plurality of holes made through its surface and extending towards the inside of the tissue, wherein said plurality of holes is made using one or more needles, wherein said holes intersect at least partially thus forming holes that communicate at least partially with each other.

2) Method according to claim 1), wherein it also comprises a step in which reservoir cavities that communicate with at least part of said holes are created in said tissue.

3) Method according to claim 1), wherein said holes and/or reservoir cavities are made by means of one or more metal needles connected to an electric power source, causing on the tip of each needle the passage of a current whose intensity and wave shape are such as to supply a quantity of energy sufficient to open the bonds that join the molecules of the organic tissue in proximity to the tip of said needle.

4) Method according to claim 3), wherein the electric current is determined by a voltage with a wave frequency of 4 MHz in which the sinusoidal voltage wave applied is a distorted sinusoidal wave and therefore with harmonics at least in the first, second and third order.

5) Method according to claim 1), wherein said holes have a diameter of at least 50 μm.

6) Method according to claim 2), wherein said reservoir cavities are substantially cylindrical and have a diameter of up to 1 mm.

7) Method according to claim 1), wherein said holes have different diameters.

8) Method according to claim 2), wherein said reservoir cavities have different diameters.

9) Method according to claim 1), wherein it comprises a step in which various materials are introduced in the holes with the aid of a suction system, wherein the materials are preferably selected among washing solutions, nutrition solutions and/or living cells.

10) (canceled)

11) (canceled)

12) (canceled)

13) (canceled)

14) (canceled)

15) (canceled)
